(19) United States
(12) Patent Application Publication
(10) Pub. No.: US 2024/0412833 A1
(43) Pub. Date: Dec. 12, 2024

(54) METHODS AND SYSTEMS FOR CREATING MEDICAL REPORT TEXTS

(71) Applicant: Smart Reporting GmbH, Munich (DE)

(72) Inventors: Su Hwan KIM, Munich (DE); Francisco PINTO, Munich (DE); Sigrid AUWETER, Puchheim (DE); Sonja SELDER, Munich (DE)

(21) Appl. No.: 18/698,905

(22) PCT Filed: Oct. 8, 2021
(86) PCT No.: PCT/EP2021/077927

§ 371 Date: Apr. 5, 2024

(51) Int. Cl. G16H 15/00; G06F 40/169

(52) U.S. Cl. CPC G16H 15/00; G06F 40/169

(57) ABSTRACT

Methods, systems and computer program products for creating a medical report indicative of a follow-up examination of a patient are provided. The method comprises receiving a selection of at least one reference report text of the patient, wherein the reference report text has at least one reference data element associated with a mark-up element; creating a partial follow-up report text, having at least an empty data element, associated with the mark-up element; displaying the follow-up report text; displaying a call-to-action element based at least on the received selection of reference report text, the call-to-action element requesting input for the empty data element; receiving an input element in response to the displayed call-to-action element; and creating the follow-up report text based on the partial follow-up report text and the received input element, wherein the created follow-up report text has at least the input element associated with the mark-up element.

### FIELD OF THE INVENTION

This invention relates to the field of creating medical report texts.

### BACKGROUND OF THE INVENTION

Medical reports are drafted by physicians for a variety of medical examinations and procedures. Accurate and detailed documentation is important, for instance for medicolegal reasons as well as for sharing and distributing relevant clinical information to medical staff involved in the care of the respective patient.

When drafting a medical report, e.g., of a patient's diagnostic examination, physicians summarize their observations made and the conclusions drawn as well as actions to be followed (e.g., further diagnostics, recommendations). Depending on the nature of the observations and of the conclusions drawn, many patients will, after a given time, return for a follow-up examination, which is to ascertain the temporal development of the medical condition. As a result, multiple medical reports for a given patient may be created over the course of time.

Upon such follow-up examination, a physician (who may be the same individual that has performed the previous examination or who may be another individual) will again draft a medical report on the follow-up examination, i.e., a medical follow-up report. The content of previous report may inform the creation of any subsequent report of the patient.

Various approaches have been suggested to create a follow-up report. Conventionally, the follow-up report may have been created completely anew, i.e., independently from any previous report. Such approaches have led to the creation of isolated documents for each examination, with diverging structures and thus impaired comparability between the previous report and the follow-up report or analyzability over the course of the medical history of the patient.

Another approach for creating a follow-up report may have been for the follow-up physician to entirely copy-paste a previous report and manually parse through the text to identify portions of the text which warrant deletion or changes. However, such approaches are prone to errors in that the physician might overlook portions to be updated such that the follow-up report does not reflect the actual findings of the follow-up examination leading to misinterpretations.

Against this background, there is a need to improve the creation of medical reports indicative of a follow-up examination of a patient.

### SUMMARY OF THE INVENTION

The invention provides methods, systems, and computer program products to improve the creation of medical report texts indicative of a follow-up examination of a patient. Aspects of the invention are defined in the independent claims. The dependent claims relate to preferred embodiments.

One aspect of the invention concerns a method for creating a medical report, the medical report being indicative of a follow-up examination of a patient. The method comprises receiving a selection of at least one reference report text of the patient. The reference report text has at least one reference data element associated with a mark-up element.

For instance, the mark-up element may be a hierarchical structure element indicative of a location within a hierarchical structure. Additionally, or alternatively, the mark-up element may be an annotation element indicative of a medical annotation.

Generally, the method further comprises creating a partial follow-up report text, having at least an empty data element, associated with the mark-up element, and displaying the follow-up report text.

Furthermore, the method comprises displaying a call-to-action element based at least on the received selection of reference report text, the call-to-action element requesting input for the empty data element.

Further, the method comprises receiving an input element in response to the displayed call-to-action element, and creating the follow-up report text based on the partial follow-up report text and the received input element. The created follow-up report text has at least the input element associated with the mark-up element.

Another aspect of the invention concerns a computer program product for creating a medical report, the medical report being indicative of a follow-up examination of a patient, which is stored on a non-volatile storage medium and contains computer-readable instructions for carrying out the steps of a method according to the first aspect.

Another aspect of the invention concerns a computer system for creating a medical report, the medical report being indicative of a follow-up examination of a patient. The system comprises a processing unit, an input receiving unit to receive input from a user and to communicate said input to the processing unit, and a displaying unit configured to display information to the user.

The processing unit is configured to perform the steps of a method according to the first aspect.

The above and other advantages result from the following considerations, where aspects and embodiments of the invention are discussed and, where appropriate, reference is made to the corresponding drawings, which show preferred embodiments of the invention for illustration purposes. However, these embodiments do not necessarily represent the full scope of the invention.

### BRIEF DESCRIPTION OF THE DRAWINGS

FIG. 1 shows a schematic illustration of the steps of a method according to the invention;

FIG. 2 shows a schematic illustration of a first example of a displayed partial follow-up report text associated with a mark-up element;

FIG. 3 shows a schematic illustration of an example of a reference report with a hierarchical structure for use in the invention;

FIG. 4 shows a schematic illustration of an example of a partial follow-up report with a hierarchical structure for use in the invention;

FIG. 5 shows a schematic illustration of a second example of a displayed partial follow-up report text;

FIG. 6 shows a schematic illustration of the partial follow-up report text associated with a mark-up element according to the second example; and

FIG. 7 shows a schematic illustration of a system according to the invention.

### DETAILED DESCRIPTION

The order of any of the steps described in the following can be changed where appropriate. Furthermore, the steps can be performed individually or in combination with each other, where appropriate. All the method steps described can be performed or implemented on a computer system.

One aspect of the invention concerns a computer-implemented method for creating a medical report, the medical report being indicative of a follow-up examination of a patient.

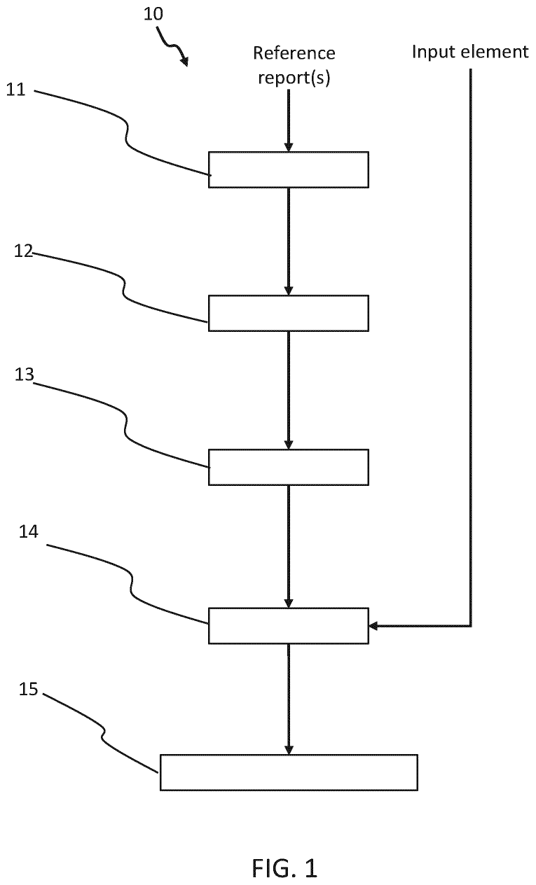

FIG. 1 shows an embodiment of a method 10 according to said aspect. According to the embodiment illustrated in FIG. 1, a selection of one or more reference report texts of the patient is received at step 11. The reference report text has at least one reference data element associated with a mark-up element. In particular, the mark-up element may mark the at least one reference data element as requiring input in a follow-up examination.

In some embodiments, the mark-up element may be a hierarchical structure element. This may in particular be useful in the case of structured reference report texts.

Structured report texts comprise the actual text along with or embedded in a hierarchical structure, such as a hierarchical graph. Contrary to free-text reports, so-called structured reports are known in the art. Structured reports may be machine-readable and may have an at least partially standardized structure. As a result, they may contain the same elements, wording, and layout as other comparable structured reports. For the generation of structured report, pre-defined report templates can be used, which provide case-specific structure and include recommended reporting steps. Alternatively, a user may select predefined elements, which are then combined to form a report. Further approaches provide for a parsing of a free-form text in order to recognize a structure and mark it accordingly.

Examples of such structures include a hierarchical graph, such as a tree. The nodes (root node, internal nodes and leaf nodes) of such tree are examples of hierarchical structure elements. Thus, in case the reference report text is structured, various elements (nodes, leaves) may be marked as requiring input in a follow-up examination. Such marking as requiring input in a follow-up examination may for instance be included in the afore-mentioned pre-defined report templates.

Whether or not a particular element requires follow-up input may depend on the type or nature of the information encoded therein. Examples of data which require follow-up input are data which typically change over time. For instance, the size of a tumor or the severity of a lung infiltration or a test result (e.g., blood oxygen concentration) typically will change from the previous examination to the follow-up examination. Structure elements encoding such data may thus be marked as requiring input in a follow-up examination. Such marking may be implemented using a binary marker (e.g., true/false flag). In other examples, it may be implemented using a parameter, such as time threshold, which indicates the duration of time after the previous examination (e.g., in number of days, weeks, months, or years after the previous examination) after which the reference data element requires new input (whereas within that duration, no input is required for this reference data element).

Other data, which typically do not change over time, may not be marked as requiring input in a follow-up examination. Alternatively, they may be marked as being constant over time. Examples of such data include the date of birth of the patient or any prior medical history (e.g., date and type of an organ transplant).

As will be apparent to the skilled person from the following description of preferred embodiments described herein, the mark-up element is not restricted to being a hierarchical structure element. For instance, additionally or alternatively, the mark-up element may be an annotation element indicative of a concept of a medical ontology.

This may in particular be useful in the case of annotated reference report texts. Annotated report texts comprise the actual text along with a pointer to or indication of a medical concept in a medical ontology.

Such medical ontology may be specific to a given medical domain, e.g., pulmonology, or it may be a general ontology covering many domains. For instance, databases representing standardized medical ontologies, such as SNOMED CT or ICD-10, may be used. Further examples include “Logical Observation Identifiers Names and Codes” (LOINC) in the field of laboratory medicine, RxNorm in the field of clinical drugs, RadLex in the field of radiology, or “Infectious Disease Ontology” (IDO) in the field of infectious diseases. Such databases may include a vast number of entries (nodes) and relations, such as hundreds of thousands of entries in the current version of SNOMED CT. Furthermore, the content of the knowledge bases to be used may be dynamically updated to reflect the expansion of medical knowledge.

An annotation of a report text allows to enrich the actual text (which is reflective of a particular patient) with more fundamental relations taken from medical knowledge. The concepts included in such annotation may be marked as requiring input in a follow-up examination or may be marked as not requiring input in a follow-up examination. Such annotation may be based on medical ontologies, with supplementary information. For instance, a pulmonology ontology database may reflect the medical meaning of and relationship between concepts in the field of pulmonology.

In general, such medical ontologies may be extended with supplementary information to thereby indicate that a given concept may be susceptible to changes from one examination to the next and thus to require input in follow-up examination. For instance, returning to the example of an ontology in the field of pulmonology, the concept of severity of lung carcinoma may be marked in the supplementary information (e.g., an extension to the ontology) to the effect that this concept requires input in a follow-up examination, whereas the location of such tumor (lung) is not an annotation requiring input in a follow-up examination.

Therefore, similar to the above, the marking of a concept as requiring input in a follow-up examination may be implemented using a binary marker (e.g., true/false flag). In other examples, it may be implemented using a parameter, such as time threshold, which indicates the duration of time after the previous examination (e.g., in number of days, weeks, months, or years after the previous examination) after which the reference data element requires new input (whereas within that duration, no input is required for this reference data element).

Thus, annotated reference report texts may be provided with mark-up element by reference to the underlying medical ontology.

In some embodiments, the received selection may comprise multiple reference reports. In some examples of such embodiment, the multiple reference reports may have a common structure. In other examples of such embodiment, a first reference report from the multiple reference reports has a first structure and a second reference report from the multiple reference reports has a second structure, the first and second structures not being identical.

Returning to FIG. 1, method 10 further comprises a step 12 of creating a partial follow-up report text, having at least an empty data element, associated with the mark-up element. For instance, the creating may comprise creating a report based on those contents of the reference report text which are not marked as requiring input in a follow-up examination. In particular, such creating may comprise a copying of those contents. In other examples, the partial follow-up may be created using text indicating that these attributes have not changed (such as generation of text “size and location remain unchanged relative to the afore-described report”).

The method 10 further comprises a step 13 of displaying. In the example shown, the step 13 comprises both a displaying of the follow-up report text and displaying of a call-to-action element. In other examples, these two elements may be displayed non-simultaneously. In particular, first the follow-up report text may be displayed and then this display may subsequently be complemented (or replaced) by the display of the call-to-action element. The call-to-action element is based at least on the received selection of reference report text and requests input for the empty data element.

In some embodiments, the call-to-action element may comprise or be a graphical instruction and/or an audio instruction. Examples of a graphical instruction include a pop-up window, a color highlighting, a pictogram or an icon. Examples of an audio instruction include a signal beep or a speech instruction. This allows that all empty data elements are effortlessly locatable by the user to ensure that the follow-up report text is created without omissions of any data element requiring input in the follow-up examination.

In some embodiments, the call-to-action element may further be configured to indicate the value of the reference data element. This allows to provide context to the user as to the previously reported value or description of the data element in question. In the case of a numerical value, it also reminds the user of the unit used for indicating the value (e.g., whether blood oxygen level was indicated in units of “mm Hg” or as a saturation in units of percentage points) such that the same unit may be used by the user for indicating the input element in the follow-up examination. Similarly, in the case of a descriptive value, it may remind the user of the terminology used for indicating the value of the reference data element in the previous examination (e.g., which of the terms “average” or “medium” or “normal” was using for indicating the blook oxygen level in the previous examination), such that the user may adhere to the same terminology for indicating the input element in the follow-up examination.

Furthermore, indicating the value of the reference data element may allow for facilitating the user's access to this information and thereby inform the medical diagnosis and/or the creation of the follow-up report.

In some embodiments, the call-to-action element may further be configured to indicate quick-action options for inputting the required input element. For instance, multiple options (e.g., each associated with a short-cut key) may allow for a rapid and easy input of the input element to be considered for the follow-up report.

Method 10 further comprises a step 14 of receiving an input element in response to the displayed call-to-action element. In some embodiments, the receiving of the input element may comprise receiving one or more of the following: one or more keystrokes from a keyboard device; one or more touch commands from a touch-sensitive device; one or more clicks from a pointing input device; a vocal command from an audio capture device; an input command from a virtual reality device or augmented reality device.

Method 10 further comprises a step 15 of creating the follow-up report text based on the partial follow-up report text and the received input element. The created follow-up report text has at least the input element associated with the mark-up element.

In some examples, the method further comprises a step of generating a graphical or textual indication, which is indicative of a temporal evolution. The temporal evolution may comprise at least the reference data element and the input element. The generation (and display) of a graphical or textual indication indicative of the temporal evolution allows to facilitate the physician's access to this information and reduces the risk for error in manual (or even mental) calculation or estimation of a trend. It thereby informs the medical decision making and the creation of the follow-up report.

An example of a graphical indication may include a graph showing the evolution of a quantitative measure as a function of time. Other types of data, including non-quantitative measures such as localization, may also be indicates graphically.

An example of a textual indication may include a summary of a temporal trend (e.g., “the size of the tumor has increased by 10%” or “the location of the lung infiltrate has moved from right upper lobe to right lower lobe”).

In some embodiments, the call-to-action element may further be configured to display one or more multimedia elements (e.g., picture element, video element, audio element) associated with the reference data element. Such multimedia elements may provide context for the user as to the medical history of the data element in question.

For instance, the one or more multimedia elements may comprise a picture element in the form of a graph indicative of a temporal evolution of the reference data element. For instance, in the case of multiple reference report texts, wherein each reference report includes a respective reference data element indicative of a given parameter (e.g., size of the lung cancer), the graph may indicate the temporal evolution (of the size of the lung cancer, for instance) over the course of time spanning all reference reports. Each of the multiple reference report texts may correspond to one data point (associated with the point in time when the respective reference report was created or when the respective reference examination was carried out). This allows to provide context for the user as to the medical history of the data element in question and thereby inform the progression or regression of the medical condition.

Additionally, or alternatively, the one or more picture elements comprises a medical image recording associated with the reference data element in the at least one selected reference report text. For instance, in the case of a report in the field of radiology, the reference report may be associated with one or more radiology image recordings. Furthermore, the follow-up examination may involve recording and assessing a medical imaging of similar type. By providing the medical image recording associated with the reference data element, the user is provided with context to assess the follow-up medical imaging. Similarly, video elements or audio elements may also be associated with the reference data element.

In such cases of displaying a medical image recording, the method may further comprise loading the medical image recording element in response to the reception of the selection. This allows to reduce the processing time in the creation of the follow-up report.

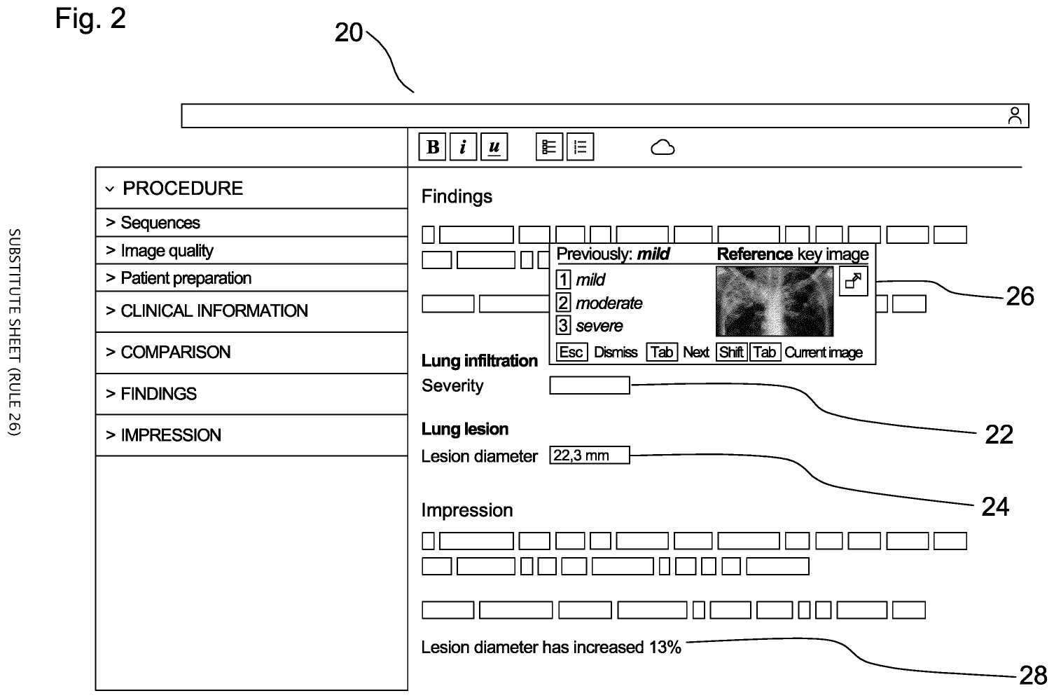

FIG. 2 shows a schematic illustration of an example of a computer display 20 showing a partial follow-up report text associated with a mark-up element.

In the example shown, one structured reference report text had previously been selected and received, such that the partial follow-up report shown in FIG. 2 has been created. Initially, the partial follow-up report has had two empty data elements, one concerning the severity of lung infiltration and one concerning the lesion diameter of the lung lesion.

In the display 20 shown in FIG. 2, the user has already provided input in the (partial) follow-up report text for one of these two empty data elements, namely for the current lesion diameter. At the time of the follow-up report, the current lesion diameter 24 is indicated as “22.3 mm”. As a result, the display 20 includes a textual indication 28 (“lesion diameter has increased 13%”) generated in response to the input of the current lesion diameter (22.3 mm) in comparison to the corresponding reference data element (not shown). The textual indication 28 is indicative of a temporal evolution. Such generation of temporal evolution indicators reduces the risk for error in manual (or even mental) calculation or estimation of a trend.

Contrary to the input element of lesion diameter, in the display 20 shown in FIG. 2, the user has not yet provided input in the (partial) follow-up report text for the other of these two empty data elements, namely for the current severity of lung infiltration. As a result, display 20 includes multiple call-to-action elements 22, 26 generated based on the received selection of reference report text and requesting input for the empty data element concerning current severity of lung infiltration.

As a first call-to-action element, the display 20 includes a graphical instruction 22 in the form of color highlighting immediately adjacent to the description “severity” in the “lung infiltration” subsection. For instance, a red color highlighting may be indicative of required input.

Once input has been received, the color highlighting may disappear or may change color (e.g., from red to green), the changed color (e.g., green) being indicative of a received input. Such changed color highlighting is shown in FIG. 2 for the example of the lesion diameter mark-up element.

As a second call-to-action element, the display 20 includes a graphical instruction 26 in the form of a pop-up window, adjacent to the field where input is required or expected. In some examples, the pop-up window may be displayed constantly (from creation of the partial follow-up report until completion of the follow-up report or at least until reception of input for that empty data element). In other examples, the pop-up window may be displayed temporarily, e.g., upon selection of the empty data element (such as when placing a text input cursor in the empty data field or when hovering over the empty data field with a pointing device).

The pop-up window 26 comprises a variety of information indicators: At the top left, on indicator relates to the value of the reference data element (“previously: mild”). This allows the user to immediately recognize the previous value of severity of the lung infiltration at the time of the selected reference report.

Furthermore, another indicator of the pop-up window relates to quick-action options for inputting the required input element. In the example shown, the pop-up window provides three options (“mild”, “moderate”, and “severe”), each associated with a short-cut key (number keys 1, 2, and 3, respectively), which allows for a rapid and easy input of the input element to be considered for the follow-up report. Other short-cut interface may involve dictation devices or enhanced reality devices.

Furthermore, another indicator concerns the display of a multimedia element associated with the reference data element. In the example shown, a picture element as an exemplary multimedia element is a medical image recording (radiology image) associated with the reference data element “lung infiltration” as recorded in the context of the selected reference report text. Such multimedia or picture elements may provide context for the user as to the medical history of the data element in question, e.g., to further assess the previous severity of the lung infiltration.

In other examples, only one of these call-to-action elements (or any other call-to-action element) may be displayed.

In further examples (not shown), the pop-up window may comprise further displays providing context for the user as to the data element in question. For instance, it may display general clinical background information concerning the findings and highlight which type of information is typically specified by the user. It thereby facilitates the user's entry of a complete follow-up report.

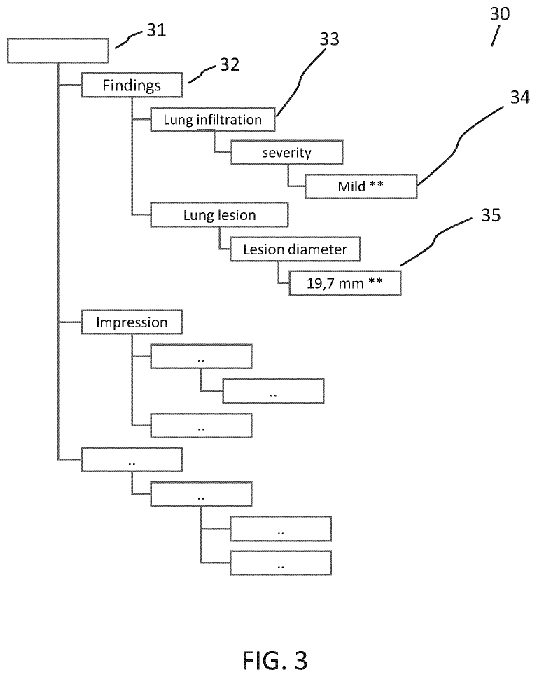

FIG. 3 shows a schematic illustration of an example of a hierarchical structure 30 of a reference report text for use in the invention.

The hierarchical structure 30 shown in FIG. 3 corresponds essentially to the reference report text selected for the partial follow-up report shown in FIG. 2.

In particular, hierarchical structure 30 comprises a plurality of nodes, connected by edges. Examples of these nodes include root node 31, internal nodes (such as nodes 32 and 33) as well as leaf nodes (such as nodes 34 and 35). Each of the nodes is associated with a corresponding text or data element. In the example shown, two of the nodes (namely leaf nodes 34 and 35) are mark-up elements as illustrated by virtue of two asterisks ** in FIG. 3. This means that the text elements (“mild” and “19.7 mm”) corresponding to mark-up elements 34 and 35 require input in a follow-up examination.

In the example shown, the classification of leaves 34 and 35 as mark-up elements had been included in the pre-defined template used for generating the reference report. In other examples, such classification may be generated based on other or further criteria. Examples of other such criteria include that any leaf node may be classified as mark-up element. Additionally, or alternatively, a classification algorithm may be used. Examples of classification algorithms include machine learning algorithms which are trained on a set of manually classified structured reports or templates.

In other examples, not shown, the classification of elements as mark-up elements may also be applied to other structures than hierarchical structures. As mentioned above, annotation elements indicative of a concept of a medical ontology may be used as mark-up elements. In such cases, the concepts of the medical ontology may be classified as requiring input in a follow-up examination (i.e., be classified as mark-up elements). Such classification may preferably be carried out using a classification algorithm as mentioned above. The classification algorithm may in particular be a machine learning procedure trained on a set of annotated reports, where corresponding elements of multiple reports are annotated using a same concept from the ontology. By detecting which of these elements show a temporal evolution (i.e., which are changed from a previous report to a subsequent report), the procedure may learn that any element associated with that same concept from the ontology shall be classified as requiring input in a follow-up examination.

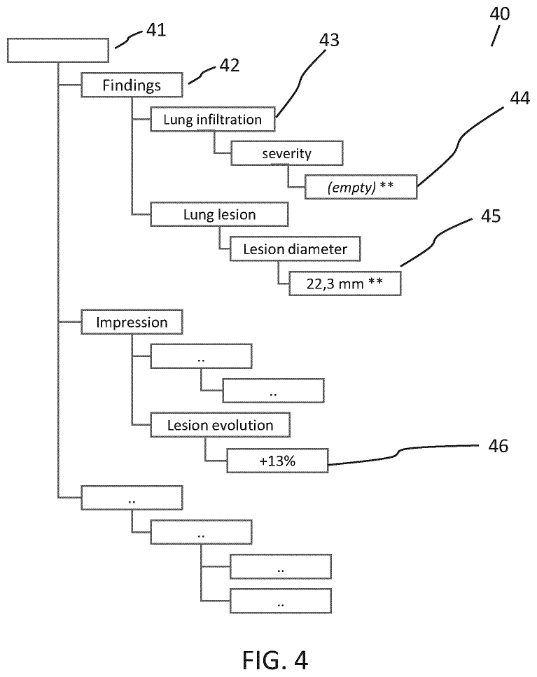

FIG. 4 shows a schematic illustration of an example of a hierarchical structure 40 of a partial follow-up report for use in the invention.

The hierarchical structure 40 shown in FIG. 4 corresponds essentially to the partial follow-up report text created and shown in FIG. 2.

Similar to hierarchical structure 30, hierarchical structure 40 comprises a plurality of nodes, connected by edges. Examples of these nodes include root node 41, internal nodes (such as nodes 42 and 43) as well as leaf nodes (such as nodes 44, 45 and 46). Most of these nodes are associated with a corresponding text or data element. Two of the nodes (leaf nodes 44 and 45) are mark-up elements as illustrated by virtue of two asterisks ** in FIG. 4. This means that these require input in the follow-up examination.

In the example shown, leaf node 44 is associated with an empty data element, whereas leaf node 45 is (already) associated with a received input element.

Furthermore, responsive to the reception of input element corresponding to leaf node 45 (“22.3 mm”), the system has generated a further element 46, which indicates the temporal evolution of the lesion diameter elements relative to the reference report. Such generation is allowed by the fact that both the reference report and the follow-up report share a same structure and that the sequence of reference element 35 and input element 45 are located at the same location within the structure. The value of the temporal evolution (“+13%”) reflects the increase in lesion diameter (from 19.7 mm to 22.3 mm).

In some examples, the method may further comprise generating support information, which provides medical context to the temporal evolution. For instance, depending on the value of the temporal evolution, the system may recommend specific conclusions (e.g., on subsequent examinations or treatments). For instance, a slowly progressing or regressing medical condition may warrant a different examination or treatment than a rapidly progressing condition. The systems described herein provide for a reliable way to assess automatically assess temporal evolutions from one or more reference reports to a follow-up report and thereby allow to systemize the decision-making process.

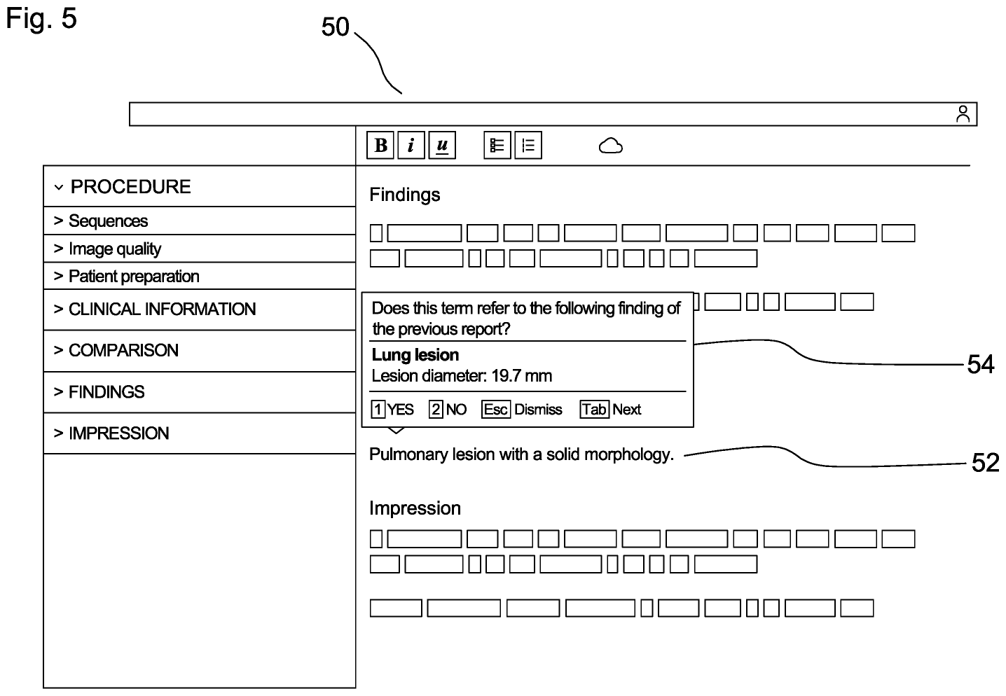

FIG. 5 shows a schematic illustration of another example of a computer display 50 showing a partial follow-up report text, created upon selection of a reference report text. In particular, the selected reference report may be the structured reference report text described above with reference to the hierarchical structure of FIG. 3.

Initially, the partial follow-up report has had at least one empty data element. The user has then provided a free-text entry of text element 52. In particular, the user entered (in the partial follow-up report text) that the findings relate to a “pulmonary lesion with a solid morphology”.

As can be seen from FIG. 3, the reference report includes data elements relating to a lung lesion, in particular having a lesion diameter of 19.7 mm associated with leaf node 35 in FIG. 3.

Returning to FIG. 5, the method according to the present disclosure may comprise a step of receiving input establishing an association between the received user input and the reference data element. For instance, the follow-up report may be parsed (e.g., in real-time as it is being entered) for text elements which are to be associated with data elements of the reference report.

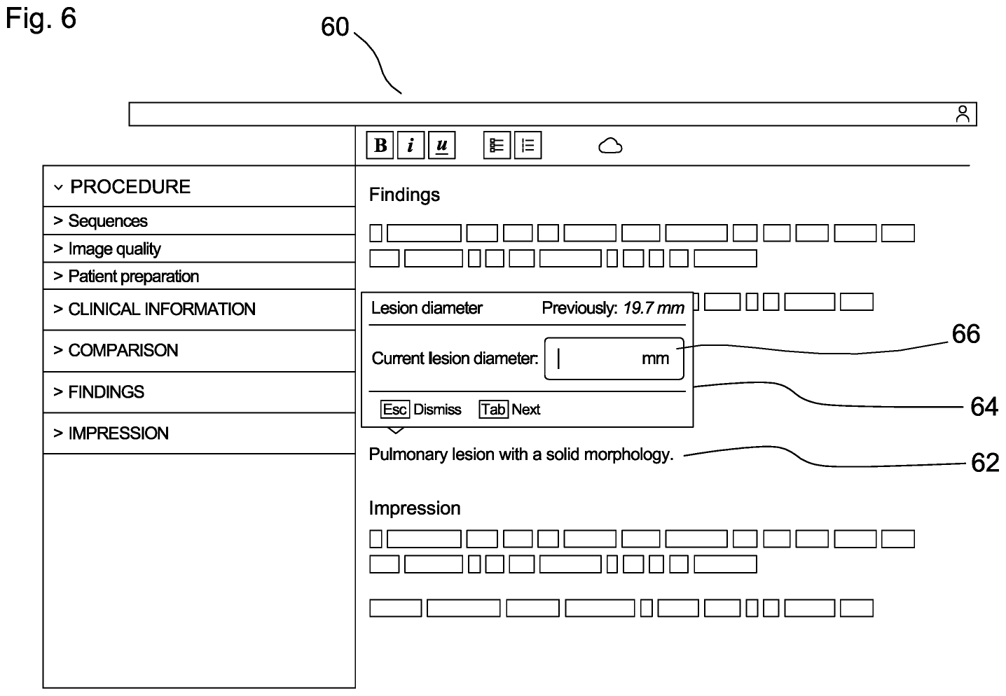

In the example of FIG. 5, the user has entered the term “pulmonary lesion”. By reference to annotations from a medical ontology, this term may be understood as being synonymous with inter alia “lung lesion”. As a result, the method displays a prompt 54 to the user to confirm that the association with a particular data element of the reference report is correct. The prompt 54 comprises a reproduction of the data elements of the reference report. Upon confirmation by the user (e.g., using short-cut keys), the free-text entry of the follow-up report is being associated with the reference data element. This association allows to place the free-text entry structurally in the same portion of the structure. An exemplary subsequent display is shown in FIG. 6.

FIG. 6 shows a schematic illustration of a computer display 60 showing the partial follow-up report text of FIG. 5 along with a call-to-action element 64, displayed upon user input in the follow-up report text. Call-to-action element 64 is based not only on the received selection of reference report text, but also on the established association between the received user input and the reference data element.

In particular, the user entry of text 62 referring to “pulmonary lesion” has established an association with mark-up element 35 of the reference report text. The mark-up element 35 relates to the lesion diameter of 19.7 mm in the reference report and indicates that this element requires input in the follow-up examination.

As a result, computer display 60 includes the call-to-action element 64 requesting input for the empty data element concerning current lesion diameter of the lung lesion.

The call-to-action element 64 of display 60 includes a graphical instruction in the form of a pop-up window, e.g., adjacent to the free-text input. The pop-up window 64 comprises a variety of information indicators: At the top right, one indicator relates to the value of the reference data element (“previously: 19.7 mm”). This allows the user to immediately recognize the previous value of diameter of the lung lesion at the time of the selected reference report

Furthermore, another indicator of the pop-up window relates to quick-action options for inputting the required input element. In the example shown, the pop-up window provides a text-entry field 66, which allows for a rapid and easy input of the input element to be considered for the follow-up report.

The association of received user input with reference data elements allows to establish a relationship even in cases where the attributes of the reference report text have only partially been annotated as requiring input in a follow-up examination.

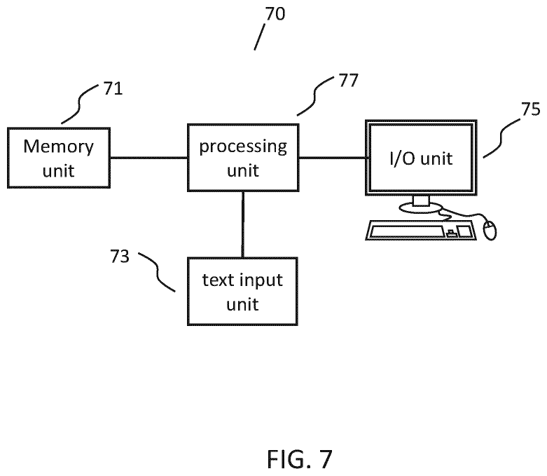

FIG. 7 shows a schematic illustration of a system 70 according to the invention. System 70 comprises an input receiving unit 73, an output unit 75 and a processing unit 77.

The input receiving unit 73 is configured to receive input from a user and to communicate said input to the processing unit. For instance, it may receive text, for instance from a typing device (keyboard, touch screen, etc.), from an audio recording device (microphone, dictating device, etc.) or from a picture recording device (camera, scanner).

Output unit 75 is configured to display or otherwise output information to the user.

The processing unit 77 is configured to obtain the input from the input receiving unit, to control the output unit 75 and to carry out any of the method steps described above. The processing unit 77 can, e.g., consist of a single- or multi-core processor or multiple physical processors (also referred to as multiple sockets).

In some embodiments, system 70 further comprises a memory unit 71. The memory unit 71 is configured to store one or more reference report texts. Optionally, it may also store the created follow-up report text. In some examples, the memory unit may be implemented as a single device. In other examples, the storing of reference report texts and the storing of the follow-up report text may be functionally separated. For ease of illustration, in FIG. 7, a single memory unit is shown.

What is claimed is:

1. A method for creating a medical report, the medical report being indicative of a follow-up examination of a patient, the method comprising
receiving a selection of at least one reference report text of the patient, wherein the reference report text has at least one reference data element associated with a mark-up element;
creating a partial follow-up report text, having at least an empty data element, associated with the mark-up element;
displaying the follow-up report text;
displaying a call-to-action element based at least on the received selection of reference report text, the call-to-action element requesting input for the empty data element;
receiving an input element in response to the displayed call-to-action element; and
creating the follow-up report text based on the partial follow-up report text and the received input element, wherein the follow-up report text has at least the input element associated with the mark-up element.

2. The method according to claim 1, wherein the mark-up element is at least one of the following:
a hierarchical structure element indicative of a location within a hierarchical structure; or
an annotation element indicative of a medical annotation.

3. The method according to claim 1, further comprising:
receiving user input in the displayed follow-up report text; and
establishing an association between the received user input and the at least one reference data element,
wherein the displaying of the call-to-action element is further based on the established association.

4. The method according to claim 1, further comprising: generating a graphical or textual indication, indicative of a temporal evolution, the evolution comprising at least the reference data element and the input element.

5. The method according to claim 1, wherein the received selection comprises multiple reference reports.

6. The method according to claim 5, wherein the multiple reference reports have a common structure.

7. The method according to claim 5, wherein a first reference report from the multiple reference reports has a first structure and a second reference report from the multiple reference reports has a second structure, the first and second structures not being identical.

8. The method according to claim 1, wherein the call-to-action element comprises a graphical instruction or an audio instruction.

9. The method according to claim 1, wherein the call-to-action element is further configured to indicate the value of the reference data element.

10. The method according to claim 1, wherein the call-to-action element is further configured to display one or more multimedia elements associated with the reference data element.

11. The method according to claim 10, wherein the one or more multimedia elements comprise a picture element of a graph indicative of a temporal evolution of the reference data element.

12. The method according to claim 10, wherein the one or more multimedia elements comprise a picture element of a medical image recording associated with the reference data element in the at least one selected reference report text.

13. The method according to claim 12, further comprising: loading, responsive to the reception of the selection, the medical image recording element.

14. The method according to claim 1, wherein the mark-up element is a hierarchical structure element.

15. The method according to claim 1, wherein the mark-up element is an annotation element indicative of a concept of a medical ontology.

16. The method according to claim 1, wherein receiving of the input element comprises receiving one or more of the following:
one or more keystrokes from a keyboard device;
one or more touch commands from a touch-sensitive device;
one or more clicks from a pointing input device;
a vocal command from an audio capture device; or
an input command from a virtual reality device or augmented reality device.

17. A computer program product for creating a medical report, the medical report being indicative of a follow-up examination of a patient, which is stored on a non-volatile storage medium and contains computer-readable instructions that, when executed by a processor, cause the processor to carry out steps comprising:
receiving a selection of at least one reference report text of the patient, wherein the reference report text has at least one reference data element associated with a mark-up element;
creating a partial follow-up report text, having at least an empty data element, associated with the mark-up element;
displaying the follow-up report text;
displaying a call-to-action element based at least on the received selection of reference report text, the call-to-action element requesting input for the empty data element;
receiving an input element in response to the displayed call-to-action element; and
creating the follow-up report text based on the partial follow-up report text and the received input element, wherein the follow-up report text has at least the input element associated with the mark-up element.

18. A computer system for creating a medical report, the medical report being indicative of a follow-up examination of a patient, the system comprising:
a processing unit;
an input receiving unit to receive input from a user and to communicate said input to the processing unit; and
a displaying unit configured to display information to the user,
wherein the processing unit is configured to:
receive, with the input receiving unit, a selection of at least one reference report text of the patient, wherein the reference report text has at least one reference data element associated with a mark-up element;
create a partial follow-up report text, having at least an empty data element, associated with the mark-up element;
display, with the displaying unit, the follow-up report text;
display, with the displaying unit, a call-to-action element based at least on the received selection of reference report text, the call-to-action element requesting input for the empty data element;
receive, with the input receiving unit, an input element in response to a displayed call-to-action element; and
create the follow-up report text based on the partial follow-up report text and the received input element, wherein the created follow-up report text has at least the input element associated with the mark-up element.
